(19) United States
(12) Patent Application Publication
(10) Pub. No.: US 2009/0196914 A1
(43) Pub. Date: Aug. 6, 2009

(54) LIPOSOMAL L-CARNITINE

(75) Inventor: Hans E.J. Hofland, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: OPTIME Therapeutics, Inc., Petaluma, CA (US)

(21) Appl. No.: 12/178,139

(22) Filed: Jul. 23, 2008

Related U.S. Application Data
(63) Continuation of application No. 10/713,546, filed on Nov. 14, 2003.
(60) Provisional application No. 60/426,610, filed on Nov. 14, 2002.

(51) Int. Cl. A61K 9/127; A61K 31/205

(52) U.S. Cl. 424/450; 514/556

(57) ABSTRACT

The present invention provides liposomal L-carnitine formulations and methods for the treatment of intermittent claudication. The formulation is optimized with respect to L-carnitine transport through skin in vitro. Preferably, the formulation is a topical cream for the administration of the drug directly on the muscle group that is most affected by the poor local blood flow.

### CROSS-REFERENCE OF RELATED APPLICATIONS

The present application claims priority to U.S. Provisional Patent Application No. 60/426,610, filed Nov. 14, 2002, the teaching of which is hereby incorporated by reference in its entirety for all purposes.

### BACKGROUND OF THE INVENTION

Intermittent claudication affects over 4 million individuals in the United States. Intermittent claudication is the primary symptom of peripheral arterial disease. In peripheral arterial disease, fatty deposits build up along artery walls and affect blood circulation, primarily in arteries leading to the legs and feet. Intermittent claudication is caused by an arterial obstruction in the lower extremity due to atherosclerosis. The disease is characterized by discomfort, pain, fatigue, numbness, or heaviness that is felt in the affected limb during walking and that resolves after a few minutes of rest. Patients with intermittent claudication usually experience leg pain each time they walk a certain distance at a constant walking speed and grade. In these patients, a hemodynamically significant stenosis prevents adequate blood flow during walking. The increased pressure gradient that develops across the stenosis causes a reduction in the perfusion pressure to the working muscle. As ischemia develops, local vasodilation and further reduction in arterial perfusion pressure occur. This reduction in arterial perfusion pressure causes a metabolic environment in the leg muscles in which the demand for oxygenated blood exceeds the supply. As a result, lactic acid accumulates in the ischemic muscle and pain develops.

The focus of initial treatment of intermittent claudication is exercise along with risk factor modification. The next level of therapy targets disease that is much further progressed, i.e. to remove the thrombus that occludes the artery or surgical removal of the occluded artery. Current drug therapies are long term systemic treatments (oral administration), such as anti-coagulants that are designed to prevent progression of the disease especially to prevent stroke and myocardial infarction. Pletal® (cilostazol) is a new oral drug to treat intermittent claudication. However, cilostazol is a member of a pharmacologic class that is dangerous to people with severe heart failure and unstudied in other people. Cilostazol has been studied in people without heart failure, without evidence of harm, but much more data would be needed to determine that there is no risk at all. The most obvious pharmacological strategies would be to bring more blood volume to the affected region. However, it is not likely that vasodilators are effective since the diseased vessels are usually the larger vessels that are already fully dilated. Dilating the smaller vessels will not result in an increased blood flow to the tissue. L-Carnitine mediates long chain fatty acid transport into mitochondria, thereby increasing energy production in the muscle. Carnitine, a natural substance important to the transport of fat into the mitochondria where it is consumed to produce energy, has already shown great promise in clinical trials as oral or intravenous formulations. However, these are chronic treatments with high doses.

L-carnitine is currently on the market as an oral formulation to treat patients with an inborn error of metabolism resulting in a secondary carnitine deficiency. Propionyl-L-carnitine is not on the market, but it has been tested in clinical trials and is very promising as an oral drug to treat intermittent claudication. However, the bioavailability is poor (15%) and the treatment is chronic. The patients have to take large doses (1 gram per day) over a long period of time (weeks) to see an effect.

It is well recognized in the medical field that the most effective procedure for treating localized disease is to direct the pharmaceutical or drug agent to the affected area, thereby avoiding undesirable toxic effects of systemic treatment. Liposomes are vesicles comprised of one or more concentrically ordered lipid bilayers which encapsulate an aqueous phase. Liposomes are especially useful for topical delivery and deposition of drugs into the skin and underlying muscle tissue.

To date, there is a great need to treat acute symptoms of intermittent claudication. Surprisingly, the present invention fulfills this and other needs by providing such method and means.

### BRIEF SUMMARY OF THE INVENTION

The present invention provides compositions, methods, and kits that provide delivery of L-carnitine and L-carnitine derivatives. The invention is particularly directed towards treatment or prophylaxis of intermittent claudication associated with peripheral arterial disease. The treatment according to the invention comprises administration of a liposomal formulation of an active ingredient, wherein the active ingredient is L-carnitine or an L-carnitine derivative. Advantageously, the liposomal formulation permits topical, safe, noninvasive, and easy to use administration of L-carnitine or L-carnitine derivatives to systems where intermittent claudication causes significant medical symptoms (e.g., skeletal muscle).

In one aspect, the present invention provides a liposomal formulation comprising: a) L-carnitine; and b) a liposome. In one embodiment, the L-carnitine is acetyl-L-carnitine or propionyl-L-carnitine. In another embodiment, the liposome is an Optisome®. In another embodiment, the L-carnitine is encapsulated in the liposome.

In another aspect, the present invention provides a liposomal formulation comprising: a) an L-carnitine derivative; and b) a liposome.

In another aspect, the present invention provides a method for treating peripheral arterial disease in a mammal comprising administering a therapeutically effective amount of a liposomal formulation of L-carnitine, thereby treating peripheral arterial disease in the mammal such as a human being. In one embodiment, the disease or symptom is claudication. These and other advantages, objects and aspects will be more apparent when read with the accompanying detailed description which follows.

### DETAILED DESCRIPTION OF THE INVENTION

The present invention is generally directed towards compositions and methods for liposomal L-carnitine delivery. The invention is particularly directed towards treatment or prophylaxis of intermittent claudication associated with peripheral arterial disease. Current treatments involve high doses over a prolonged period of time. Given orally, L-carnitine has shown great promise in clinical trials, however, its bioavailability is poor. The present invention provides a more efficient and faster treatment via topical application, thus allowing direct transport into the underlying muscle tissue. The treatment according to the invention comprises administration of a liposomal formulation of an active ingredient, wherein the active ingredient is L-carnitine, L-carnitine derivative or mixtures thereof. Advantageously, the liposomal formulation permits topical, safe, noninvasive, and easy to use administration of L-carnitine or L-carnitine derivatives to systems where intermittent claudication causes significant medical symptoms (e.g., skeletal muscle).

### I. L-CARNITINE AND DERIVATIVES

Carnitine is a natural substance important to the transport of fat into mitochondria where it is metabolized to produce energy. Fatty acids are utilized as an energy source in all tissues except the brain. In skeletal and cardiac muscle they serve as the major fuel. Carnitine palmitoyl transferase I and II are involved in the transport of long-chain fatty acids through the mitochondrial membranes. Carnitine is also important in removing metabolites out of the mitochondria and eliminating them from the body. Carnitine is taken in the diet through red meats and dairy products, and is also synthesized in the body from protein during muscle breakdown.

L-carnitine (Carnitor®) is currently used in acute and chronic treatment of patients with an inborn error of metabolism that results in secondary carnitine deficiency. There are no warnings and no contra-indications as this is a drug with an excellent safety profile. Propionyl-L-carnitine stimulates energy production in ischemic muscles by increasing citric acid cycle flux and stimulating pyruvate dehydrogenase activity. The free radical scavenging activity of the drug may also be beneficial. Propionyl-L-carnitine improves coagulative fibrinolytic homeostasis in vasal endothelium and positively affects blood viscosity. Improvements in maximum walking distance (MWD) correlated positively with increased mitochondrial oxidative adenosine triphosphate (ATP) synthesis in a study in patients with peripheral arterial disease.

Oral carnitine is poorly absorbed. Only 25% of the oral dose is taken up into the body, the rest is excreted in the stool. Approximately 76% of the free circulating carnitine is eliminated in the urine. Following a dose of 1980 mg twice a day, the maximum plasma concentration level (Cmax) was 80 nmol/mL and the time to maximum concentration (Tmax) was 3.3 hours.

Intravenous carnitine is fully available as it bypasses the bowel absorption problems. For this reason it is the preferred route of administration in children in life threatening crisis. Doses of carnitine used are variable and range from 50 to 600 mg/kg/day for oral carnitine, and 25 to 300 mg/kg/day for intravenous carnitine. The recommended daily dose for adults is 990 mg/day, i.e. 3×330 mg tablets per day. Higher doses are usually used in children and adults with serious metabolic disorders during times of metabolic stress and decompensations.

Carnitine is a white powder with a melting point of 196-197° C. and is readily soluble in water, hot alcohol and insoluble in acetone. The pH of a 1:20 (w/w) solution is between 6-8. The pKa value is 3.8. The chemical structures of L-carnitine and alkyl-L-carnitine are depicted in FIG. 1.

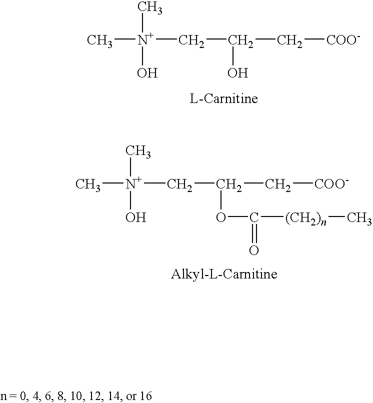

FIG. 1. Chemical Structures of L-Carnitine and Alkyl-L-Carnitine.

Suitable active ingredients of the present invention include L-carnitine, propionyl-L-carnitine, acetyl-L-carnitine, and derivatives thereof. The carbon chain length of alkyl-L-carnitine is optimized with respect to encapsulation efficiency and physico-chemical stability of the formulation.

### II. LIPOSOME SYNTHESIS AND COMPOSITION

The function of the skin is to protect the individual from the environment (chemicals, viruses and bacteria) and to protect the body from dehydrating. The stratum corneum is the outermost layer of the skin and is composed of cells embedded in lipid bilayers. The stratum corneum is known to be the main barrier for transport through the skin. Liposomes consist of lipid bilayers that are similar in composition and organization to those found in the stratum corneum. The lipid bilayers of liposomes have been shown to fuse with the lipid bilayers in the skin, thus allowing the delivery of encapsulated active compounds directly into the skin and underlying tissues, while maintaining skin integrity and avoiding significant systemic exposure.

The L-carnitine of the present invention is encapsulated in phospholipid particles, such as liposomes, nanocapsules, microparticles, microspheres, lipid particles, vesicles, and the like. Preferably, L-carnitine will be formulated in Optime's™ proprietary Optisomes™ using Optimix™ technology generally set forth in WO 00/29103, published May 25, 2000. In preferred embodiments, the L-carnitine is encapsulated or associated with liposomes.

The formation and use of liposomes is generally known to those of skill in the art (see, for example, Couvreur et al., 1977; Couvreur, 1988; Lasic, 1998; which describes the use of liposomes and nanocapsules in the targeted antibiotic therapy for intracellular bacterial infections and diseases). Liposomes with improved serum stability and circulation half-times have been developed (Gabizon & Papahadjopoulos, 1988; Allen and Choun, 1987; U.S. Pat. No. 5,741,516, specifically incorporated herein by reference in its entirety). Further, various methods of liposome and liposome-like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

In certain aspects, the liposomes of the present invention are produced using the apparatus as described in PCT Publication No. WO 00/29103, published on May 25, 2000, and incorporated herein by reference.

An apparatus is described therein that is useful for the continuous production of a composition of matter by in-line mixing. The apparatus comprises a first phase storage means capable of being maintained at a set temperature and a first pressurized transfer means for transferring the first phase from the storage means, along with an second phase storage means capable of being maintained at a set temperature and a second pressurized transfer means for transferring the second phase from the storage means. As described therein, the first phase is a lipid phase (optionally containing an active agent) and the second phase is an aqueous phase. The lipid phase storage means is capable of being maintained at a set temperature by a first temperature control means, typically within the range of about 20 to 75° C. Similarly, the aqueous phase storage means is capable of being maintained at a set temperature by a second temperature control means, typically within the range of about 20 to 75° C. The lipid phase and aqueous phase storage means are equipped with a means for continuously replenishing the lipid and aqueous phases. In this manner, the storage means function as a temperature stabilization means such that the lipid and aqueous phases are continuously fed into the storage means, where the temperature of each phase is stabilized prior to introduction into pressurized transfer means that exits each respective storage vessel.

The apparatus also has a mixing device that comprises a first metering system for receiving the lipid phase from the first pressurized transfer means, a second metering system for receiving the aqueous phase from the second pressurized transfer means, a pre-mixing system for preparing a pre-mixed formulation, a third pressurized transfer means for transferring the lipid phase from the first metering system to a first inlet orifice in the pre-mixing system and a fourth pressurized transfer means for transferring the aqueous phase from the second metering system to a second inlet orifice in the pre-mixing system. The pre-mixing system comprises a pre-mixing chamber having a first and second inlet orifice. The pre-mixing system can further comprise a means for creating turbulence in the aqueous phase prior to entry into the pre-mixing chamber.

The apparatus also has a mixer such as a static mixer for preparing a mixed formulation comprising lipid vesicles, having a mixing chamber and an optional means for determining the optical properties of the mixed formulation, a fifth pressurized transfer means for transferring the pre-mixed formulation from the outlet orifice of the pre-mixing system to the mixing chamber or other suitable connection or fitting; and an optional means for applying ultrasonic energy to the pre-mixing system, the mixing chamber or both. In a preferred embodiment, the optical properties of the mixed formulations are measured, with the means for determining the optical properties of the mixed formulation being configured so as to control the first and second temperature control means and the first and second metering systems.

The apparatus and method of the invention provide for lipid phase and aqueous phase streams that are as pulse-less as possible and are maintained at a constant pressure. This is a achieved by the precise metering systems each of which is provided with a pump that operates under positive pressure and in such a manner so as to provide precise volumetric delivery.

The mixer is preferably a static mixer, such as a laminar division type inline mixer. The mixer may have a means for controlling the temperature of the mixing chamber, which is typically within the range of about 20 to 80° C. In addition, the mixer may also have a means for controlling the degree and rate of mixing within the mixing chamber. The mixing device of the apparatus may also have a means for controlling the temperature within the open space of the mixing device, which is also typically within the range of about 20 to 80° C.

The apparatus has a dispensing means for transferring the mixed formulation from the mixing chamber into a storage chamber. This apparatus is particularly useful for the production of lipid vesicles, and more particularly multilamellar lipid vesicles. The apparatus of the invention is readily evaluated as to its particular suitability for manufacturing lipid vesicles having a pre-specified composition and configuration.

In general, liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977, 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly.

The ability to trap solutes varies between different types of liposomes. For example, MLVs are moderately efficient at trapping solutes, but SUVs are extremely inefficient. SUVs offer the advantage of homogeneity and reproducibility in size distribution. However, a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for hours or days, depending on their composition, and half lives in the blood range from minutes to several hours. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominant site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

The liposomes of this invention can be either synthesized using the desired final concentration of L-carnitine or be synthesized at a higher concentration and diluted to the appropriate concentration. The liposomes may be composed of any combination of various phospholipids and typical liposome ingredients, such as phosphatidycholine (PC), cholesterol, and stearylamine (SA). In preferred embodiments, liposomes composed of PC only or PC and SA are used to encapsulate L-carnitine. Preferably, the lipid to active ingredient ratio is such that it permits optimal encapsulation of the active ingredient. For L-carnitine-containing liposomes, the final L-carnitine concentration is preferably from about 1% to about 10% by weight, or more preferably, about 5% to about 10% by weight.

In particularly preferred embodiments, the present invention is a liposomal composition of phosphatidylcholine with about 1% to about 10% by weight L-carnitine. Advantageously, this formulation has a high encapsulation efficiency, and is not a complicated formulation as it is only comprised of one lipid.

### III. ADMINISTRATION

The L-carnitine-containing liposomes of this invention can be administered via various treatment regimens known to those of skill in the art. Preferably, the liposomes are administered topically, directly onto the skin over the muscles that are affected by the poor circulation

For topical administration to the skin and mucous membranes, the liposomal compositions may be formulated as a gel, ointment, cream, lotion, solution, suspension, spray, paste, oil, drops, or foam. The dosage forms may be formulated with mucoadhesive polymers for sustained release of active ingredients at the site of administration. The active ingredient-containing liposomes may be mixed under sterile conditions with a pharmaceutically acceptable carrier, and with any preservatives, buffers, or propellants, which may be required. Topical preparations can be prepared by combining the composition of interest with conventional pharmaceutical diluents and carriers commonly used in topical dry, liquid, cream and aerosol formulations. Ointment and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used according to the nature of the base include soft paraffin, aluminum stearate, cetostearyl alcohol, propylene glycol, polyethylene glycols, woolfat, hydrogenated lanolin, beeswax, and the like. Lotions may be formulated with an aqueous or oily base and, in general, also include one or more of the following: stabilizing agents, emulsifying agents, dispersing agents, suspending agents, thickening agents, coloring agents, perfumes, and the like. Drops may be formulated with an aqueous base or nonaqueous base also comprising one or more dispersing agents, suspending agents, solubilizing agents, and the like.

The ointments, pastes, creams and gels also may contain excipients, such as animal and vegetable fats, oils, waxes, paraffins, starch, tragacanth, cellulose derivatives, polyethylene glycols, propylene glycols, silicones, bentonites, silicic acid, talc and zinc oxide, antioxidants, alcohols, buffers, preservatives, or mixtures thereof. Sprays can additionally contain customary propellants, such as chlorofluorohydrocarbons and volatile unsubstituted hydrocarbons, such as butane and propane.

A preferred embodiment of the present invention is a liposomal cream or lotion containing the active ingredient (L-carnitine or derivatives thereof), which is directly applied to the skin over the muscles that are affected by the poor circulation. The liposomal L-carnitine cream or lotion can consist of, for example, water containing a buffer such as 200 mM Tris-HCl, (pH 7-8), a preservative such as benzalkonium chloride, up to 10% w/v 90H phospholipids that make up the liposomes, an antioxidant such as up to 1% v/v Vitamin E acetate, co-solvents 2% v/v propylene glycol, 5% (v/v) ethanol, and 1-10% L-carnitine.

It will be understood by those of skill in the art that changes in components of the drug product or changes in the manufacturer(s) or manufacturing process that may affect these parameters should be carefully evaluated for their effect on the safety, clinical effectiveness and stability of the product. If such changes are made subsequent to the preparation of the batches used in critical clinical, bioequivalence, or primary stability studies, adequate supportive comparative data should be provided to demonstrate equivalency in terms of safety, clinical effectiveness, and stability of the product.

The preferred course of therapy (e.g., dosage, frequency of dosing) can vary according to, inter alia, the mode of administration of the L-carnitine-containing liposomes, the particular liposomal composition being used, the particular disease being treated, and the particular host being treated. The optimal course of therapy for a given set of conditions can be ascertained by those skilled in the art using a conventional course of therapy determination tests and in view of the information set out herein. The effectiveness of treatment can be determined by controlled clinical trials, by methods known to those of skill in the art.

The following example is offered for illustration. It is intended neither to define nor to limit this invention in any manner. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### IV. EXAMPLES

### A. Example I

### Optimization of the Carbon Chain Length of Alkyl-L-Carnitine

This Example illustrates the identification of an alkyl-L-carnitine ester for topical delivery and to show that significant amounts of L-carnitine can be delivered to the muscle tissue by topical (dermal) administration as compared to oral administration. To enhance transdermal drug delivery, L-carnitine is formulated in a liposomal delivery system.

The encapsulation efficiency and efficiency of skin penetration is dependent on the hydrophobicity of the drug.

### Methods

The composition of the liposomal formulation is as follows: 10% w/w phospholipon 90H (Nattermann); 5% w/w propylene glycol; 5% w/w ethanol; 1% w/w vitamin E acetate; 1% w/w alkyl-L-carnitine; 0.15% benzalkonium chloride in 0.2 M phosphate buffer pH 7.0.

L-Carnitine is very hydrophilic. Therefore, a series of alkylated L-carnitine esters are purchased (Sigma/Aldrich) of increasing hydrophobicity. Alkyl chain lengths of 0, 2, 6, 8, 10, 12, 14, or 16 or 18 carbon atoms are investigated.

A widely used gel filtration technique is used to separate the free L-carnitine from liposome encapsulated, after diluting the liposomal cream 10-fold with 0.2 M phosphate buffer. L-Carnitine is quantified by HPLC analysis as described by Bieber et al. The maximum amount of L-carnitine encapsulation is correlated to the hydrophobicity of the drug.

The physical stability of the liposomal formulation is determined as follows: The liposomes are examined using light microscopy to determine a) the presence of the large multilamellar vesicles (MLVs); b) the presence of crystals or phase separation will lead to the rejection of this formulation; and c) the presence of irregular structures will be indicative of the disruption of the liposomes and will also cause the formulation to be rejected. The viscosity of the cream is determined over time using a Brookfield Instruments HBDC-VII Digital Rheometer, and will also be correlated to the hydrophobicity of the L-carnitine.

The chemical stability of the alkyl-L-carnitine esters is determined over time using HPLC analysis.

### Results

The encapsulation efficiency of a hydrophilic drug is greatly enhanced when this drug is rendered more hydrophobic (e.g. by alkylation). Typically, an alkyl chain length of 12 carbon atoms will yield an encapsulation efficiency greater than 95%. By studying a range of alkyl chain lengths from 0-18 carbon atoms, well over 95% encapsulation efficiencies is achieved. A systematic range of compounds varying only in the alkyl chain length is investigated. Therefore, it is highly likely that a range of compounds can be identified that has a high encapsulation efficiency such that maximal efficiency can be expected from these liposomal formulations.

The dilution of the liposomal cream prior to application on the gel filtration column may lead to an underestimation of the encapsulation efficiency of the more hydrophilic compounds. However, for the more interesting hydrophobic compounds this error will be negligible.

It has been shown that liposomes can greatly enhance the stability of chemically labile compounds. Therefore, alkyl-L-carnitine esters are protected from hydrolysis while they are encapsulated within the liposomal membranes. Once delivered into the muscle tissue these compounds are easily hydrolyzed into the active L-carnitine drug and a fatty acid.

### B. Example II

### Optimization of Formulation with Respect to L-Carnitine Transport Through Stratum Corneum In Vitro

This Example illustrates the optimization of formulation with respect to L-carnitine transport through stratum corneum in vitro. The stratum corneum is the outermost layer of the skin which is known to be the rate limiting barrier for transport. In vitro diffusion experiments are a good model to screen the alkyl-L-carnitine esters that have sufficient encapsulation efficiencies and that are compatible with the liposomal cream formulation, identified from experiments described in Example I. The best alkyl-L-carnitine compound is identified by its ability to diffuse through stratum corneum in vitro. Subsequently, this compound is used to optimize the liposome formulation.

### Methods

The following parameters are studied with respect to their effect on drug transport: a) alkyl chain length of the drug; b) lipid composition of the liposomes; c) amount and composition of excipients (propylene glycol, ethanol etc.) in the formulation.

In vitro permeation of L-carnitine through human stratum corneum is performed as described by Hofland et al. Briefly, human cadaver skin or pig skin is dermatomed to a thickness of approximately 0.2 mm, and placed on a filter paper soaked in 0.1% trypsin solution in PBS at 37° C., overnight. The stratum corneum is separated from the epidermis and washed with 1% anti-trypsin solution. The stratum corneum will be dried and stored in a desiccator over silica gel at room temperature until further use. Before use the stratum corneum is rehydrated and glued on a supporting membrane. Subsequently, the stratum corneum is placed in a Franz diffusion cell. The liposome suspension is placed in the donor compartment. The acceptor compartment is sampled over time and analyzed for L-carnitine content.

### Results

The rate of transport of the alkyl-L-carnitine may be dependent on:
a) The HLB of the drug. Very hydrophilic compounds are not incorporated in the liposomes and, therefore, do not benefit from the liposomal delivery system. On the other hand however, very hydrophobic drugs tend to reside on or in the stratum corneum and are not transported into the underlying muscle tissue very efficiently.
b) The composition of the liposomes. The fluidity of the membranes is one of the most important factors to determine penetration enhancement into the skin. Rigid bilayers tend to work more as a depot and retain the drug for a longer period of time, while more fluid bilayers will release the active more quickly. Bilayers are made more rigid by the incorporation of cholesterol, or membranes can be made more fluid by incorporating fatty acids such as oleic acid.
c) Excipients in the formulation. Both propylene glycol and ethanol are excipients in the “starting” formulation. The rate of transport through the stratum corneum can be modified by changing the content of these two co-solvents. Both ethanol and propylene glycol concentrations can vary between 0-20% (w/w) without disrupting the liposomes. A number of formulations have been identified that can be used for in vivo studies.

### C. Example III

### L-Carnitine Delivery into Muscle Tissues and Identification and Characterization of the Best Formulation In Vivo

This Example illustrates L-carnitine delivery into muscle tissue, and identification and characterization of a formulation in vivo. Even though the in vitro experiments are a useful tool to identify a range of formulations that are used for further development, in vivo experiments have to be performed to show that topical L-carnitine administration leads to significant accumulation of the drug into the underlying muscle tissue.

### Methods

The best formulation out of the five (5) formulations identified in Example II is determined as follows. Seven (7) groups (n=5) are tested: one (1) negative control consisting of empty liposomes, five (5) liposomal formulations containing alkyl-L-carnitine, and one (1) positive control group that receives an L-carnitine solution orally. Albino guinea pigs of 300-500 grams are used. The hair is shaved off an area of 3×3 cm on the back close to the neck. A 0.1 ml dose is applied twice a day over a period of 3 days. Immediately after application the application area is covered with Tegaderm (3M) 6×7 cm tape. The animals in the positive control group receive an oral dose of 50 mg/kg L-carnitine twice daily for 3 days. Ninety minutes after the last dose the animals are sacrificed in a CO₂ chamber. The following tissues are collected and frozen until further analysis: skin, muscle, heart, liver, lung, kidney, and blood. The skin samples are sliced horizontally with a keratotome at 0.2 mm (epidermis) and at 0.5 mm (dermis). The remaining portion of the skin is designated “subcutaneous” tissue. The L-carnitine content of the tissues is determined by HPLC according to Arakawa et al.

The second experiment is a dose response of the best formulation as identified in the previous experiment. Five groups of guinea pigs receive either 0, 1, 2, 4, or 6 applications of 0.1 ml liposomal L-carnitine. Each application is separated by 12 hours. The animals are sacrificed 90 minutes after the last application and tissues are harvested and analyzed as described above.

The final animal experiment studies the pharmacokinetics of topical L-carnitine. After application of a single dose (see above) the animals are sacrificed at the following time intervals: 0, 15, 30, 60, 90, 180, 360 minutes.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. A liposomal formulation comprising:
a) L-carnitine; and
b) a liposome.

2. The formulation of claim 1, wherein said L-carnitine is propionyl-L-carnitine.

3. The formulation of claim 1, wherein said L-carnitine is acetyl-L-carnitine.

4. The formulation of claim 1, wherein said liposome is an Optisome™.

5. The formulation of claim 1, wherein said L-carnitine is encapsulated in said liposome.

6. A liposomal formulation comprising:
a) an L-carnitine derivative; and
b) a liposome.

7. The formulation of claim 6, wherein said L-carnitine is alkyl-L-carnitine ester.

8. A method for treating a peripheral arterial disease in a mammal, said method comprising:
administering a therapeutically effective amount of a liposomal formulation of L-carnitine, thereby treating a peripheral arterial disease in said mammal.

9. The method according to claim 8, wherein said mammal is a human being.

10. The method according to claim 8, wherein said disease or symptom is intermittent claudication.

11. The method according to claim 8, wherein said L-carnitine is propionyl L-carnitine.
